(19) United States
(12) Patent Application Publication
(10) Pub. No.: US 2026/0120860 A1
(43) Pub. Date: Apr. 30, 2026

(54) USER STATE MONITORING

(71) Applicant: Arm Limited, Cambridge (GB)

(72) Inventors: Roberto LOPEZ MENDEZ, Cambridge (GB); Daren CROXFORD, Swaffham Prior (GB); Mina Ivanova DIMOVA, Great Shelford (GB)

(21) Appl. No.: 18/929,156

(22) Filed: Oct. 28, 2024

(51) Int. Cl. G16H 40/67; G16H 10/60

(52) U.S. Cl. CPC G16H 40/67; G16H 10/60

(57) ABSTRACT

A computing device and a method of operating a computing device, implementable as a computer program product, the method including first operating a first transceiver in the computing device to collect a first biosensor device data element into a storage and processing location; second operating the first or a second transceiver to collect a second biosensor device data element into the storage and processing location; applying, in the storage and processing location, machine learning model inferencing over at least the first biosensor device data element and the second biosensor device data element to derive a user condition indication for a biosensor device user; and on detecting, in the user condition indication, a predictive value above a threshold indicating a user condition requiring notification, emitting a message at an output of the computing device, where the user condition requiring notification includes one or both of a physical and mental condition.

### BACKGROUND

The present technology relates to computing processors, and particularly to methods of, and apparatus for, the operation of personal device technologies, such as mobile telephones, with biosensor devices and device applications to provide secure, intelligent monitoring and assessment of a user's condition (e.g. physical and/or mental condition) and health status.

It would be desirable to optimise the usefulness of the various data types collected by the different sensors and applications, so that a broader understanding of the user's physical and mental condition may be acquired, while retaining the confidentiality of the user's personal data.

### SUMMARY

There is thus provided according to a first approach, a method of operating a computing device comprising: first operating a first transceiver in the computing device to collect a first biosensor device data element into a storage and processing location; second operating the first or a second transceiver to collect a second biosensor device data element into the storage and processing location; applying, in the storage and processing location, machine learning model inferencing over at least the first biosensor device data element and the second biosensor device data element to derive a user condition indication for a biosensor device user; and on detecting, in the user condition indication, a predictive value above a threshold (e.g. a probability threshold) indicating a user condition requiring notification, emitting a message at an output of the computing device, where the user condition requiring notification comprises one or both of a physical and mental condition.

In a further approach, there is provided a computing device comprising: a transceiver operable to collect a first biosensor device data element into a storage and processing location; the transceiver or a further transceiver to collect a second biosensor device data element into the storage and processing location; a machine learning model inferencing engine operable in the storage and processing location to derive a user condition indication for a biosensor device user using at least the first biosensor device data element and the second biosensor device data element; and an output operable, on detecting in the user condition indication a predictive value above a threshold indicating a user condition requiring notification, to emit a message, where the user condition requiring notification comprises one or both of a physical and mental condition.

A further approach provides a computer program product stored on a non-transitory computer readable medium and comprising computer program code to: first cause a first transceiver to collect a first biosensor device data element into a storage and processing location; second cause the first or a second transceiver to collect a second biosensor device data element into the storage and processing location; apply, in the storage and processing location, machine learning model inferencing over at least the first biosensor device data element and the second biosensor device data element to derive a user condition indication for a biosensor device user; and responsive to detecting, in the user condition indication, a predictive value above a threshold indicating a user condition requiring notification, emitting a message at an output of the computing device, where the user condition requiring notification comprises one or both of a physical and mental condition.

A further approach provides a method of operating a computing device, comprising: determining in an artificial neural network a correlation between at least a first biosensor device data element, a second biosensor device data element, and a physical condition indication for a biosensor device user; and adjusting the weighting of the artificial neural network according to the correlation to establish a prediction threshold for a physical condition requiring a notification message to be emitted.

### BRIEF DESCRIPTION OF THE DRAWINGS

Various embodiments of the technology described herein will now be described, by way of example only and not by way of limitation, with reference to the accompanying drawings, in which:

FIG. 1 shows a much-simplified high-level view of an example computing system capable of performing processing according to an implementation of the present technology;

FIG. 2 shows a much-simplified view of an example of an arrangement of a personal hub technology with biosensor devices and device applications to provide secure, intelligent monitoring and assessment of a user's personal physical and/or mental status. according to an implementation of the present technology; and

FIG. 3 shows the main elements of an operating method for a personal hub technology according to an implementation of the present technology.

### DETAILED DESCRIPTION

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Other embodiments may be utilized, and structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. Directions and/or references, for example, such as up, down, top, bottom, and so on, may be used to facilitate discussion of drawings and are not intended to restrict application of claimed subject matter. The following detailed description therefore does not limit the claimed subject matter and/or equivalents.

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made. Features or limitations of various embodiments described herein, however important to the example embodiments in which they are incorporated, do not limit other embodiments, and any reference to the elements, operation, and application of the examples serve only to aid in understanding these example embodiments. Features or elements shown in various examples described herein can be combined in ways other than shown in the examples, and any such combinations is explicitly contemplated to be within the scope of the examples presented here.

The use of wearable, implantable, ingestible and other biometric sensor devices to take measurements relating to physical and/or mental characteristics and states of a human body is developing rapidly. The sensor devices may be passive data accumulator devices, or they may have active features. An example of a passive data accumulator device is a wearable step counter that accumulates a count of steps taken and displays the count. An example of a device having active features is a transdermal glucose monitor that detects deviations above and below acceptable blood sugar levels and calls a mobile phone with an alert on detecting a deviation that indicates that the patient has early signs of hyperglycemia or hypoglycaemia. Similarly, smart glasses may use an eye tracker that uses infrared light to determine eye movement, which may indicate certain blood pressure and neurological conditions.

A user may have several such biometric devices, and may also be making use of apps on a smartphone that collect data on request from a camera or other built-in detector technology. It is therefore desirable to have a way of optimising the usefulness of the various data types collected by the different sensors and apps, so that, by detecting patterns across the data from multiple devices, a broader understanding of the user's physical and/or mental condition may be acquired. Using this technology, the user can be alerted early to any potential health problems at an early stage in their development, while at the same time knowing that the data is being stored and processed with security.

Implementations of the present technology thus provide methods, apparatus and computer program products for operating a personal device in conjunction with biosensor devices and device applications to provide secure, intelligent monitoring and assessment of a user's physical and/or mental condition and health status.

While it is useful to be able to detect and respond to single indicators, such as the raised or lowered blood sugar levels discussed above, the inventors of the present technology realise the additional benefits of being able to detect combinations of physical and/or mental condition indications. In certain cases, it may be possible to detect early signs of serious physical and/or mental problems by recognising a pattern of many small indicators, in particular with reference to the user's historical baseline measurements. At the same time, the inventors realise that the information thus developed is highly sensitive and personal to the user, and thus requires a very high level of protection against undesired exposure.

Additionally, the combinations of physical and/or mental indicators may comprise a large set of very subtle anomalies that may not be immediately obvious to the casual observer, and that may never be detected by the simple, single detector systems described above. For this reason, the present technology comprises the use of an artificial intelligence system using a machine learning and inferencing (ML) model trained over a very large data set derived from a knowledge base of medical and other physical and/or mental condition data.

To meet the requirement to protect the user's data, that data may be securely stored and processed locally in a storage and processing location (e.g., hardware computer processor—CPU—and storage—RAM, for example) within a user's device, or it may be stored and processed remotely in a storage and processing location (e.g., hardware computer processor and storage) in a network-connected device, such as a server computer system, that has an equivalent level of security and integrity control. In one implementation, the storage and processing location may comprise a system-on-chip arrangement, in which data processing circuitry and data storage circuitry are arranged on a single chip. In other arrangements, portions of the processing may be performed by additional processors, such as general-purpose graphics processing units, and/or the storage may be off-chip in, for example, a bus-attached RAM unit. In all cases, the secure storage and processing of the user's data may be guaranteed by, for example, implementing an attestation and trust technology in the processor and storage arrangement.

Artificial intelligence techniques such as ML models are often used to process complex information such as text and images in training and inferencing operations. Embodiments of the present technology described herein are directed to providing analysis of data on a user device utilizing artificial intelligence, such as by using analysis architecture comprising a ML model to analyse input data including behaviour pattern data (e.g. where a change in behaviour pattern may indicate increased stress or confusion), biometric data, user routine data (e.g. where a change in routine may indicate increased stress or confusion), audio data, imagery data and other types of data to provide an output as will be described below.

The types of biometric devices that can be worn, implanted, ingested or otherwise closely collocated with the user's body include an increasing number of physical condition, mental condition and health or medical sensors. Examples include, but are not limited to, “smart” watches/wristbands/glasses (e.g., VR/AR glasses), “smart” clothing, shoes and jewellery, transdermal devices (e.g. glucose monitors), electronic implants, ingestible devices, and body cavity devices (such as gumshields, contact lenses or in-ear devices, e.g., ear buds or in-ear temperature sensors). Furthermore, devices in a user's environment may provide data, such as devices with cameras (e.g. in the user's home, work, vehicle or gym), devices with microphones (e.g. e.g. in the user's home, car etc.), gym equipment at a user's gym facility.

Many mobile (cellular) telephones are now “smart” and thus capable of supporting apps that can also be used to supply data, which may comprise image data, sound data, and motion and location/travel data, all of which may also be useful as input to an assessment of the user's physical and/or mental state and health status. The on-device sensors can also be used as an analytical tool. For example, the mobile camera can take pictures of the skin, iris, teeth, etc. and then using the present technology can provide some analysis of the user's physical and/or mental status based on image analysis of the picture. The microphone can potentially be used to analyze the heartbeat, and may also produce some sound and receive the reflected waves, possibly even in the ultrasound region of the spectrum. The inbuilt inertial measurement unit (IMU)—gyroscope and accelerometer—could also be used to register user's balance. In another possible implementation, instead of a smartphone, another type of device may be enhanced to support the present technology. For example, there may be sufficient processing power, memory capacity and communications capability for a device such as an augmented reality headset or glasses to act as a central device for operation of the present technology. Similarly, with advances in implantable devices, one such may be enhanced to support the present technology. These are merely example implementations—it will be clear to one of ordinary skill in that art that, in the Internet of Things, any suitable device, portable or static, may be adapted to incorporate the present technology.

The table here shows some examples of physical state and/or mental state information that can be derived using combined information from multiple sensors.

| | Interpreted health and |
|---|---|
| Sensor measurements | wellness insight |
| Movement, temperature, heart rate | Sleep and mental readiness |
| Heart rate, skin conductance, tremor, | Stress levels |
| cortisol | |
| Heart rate, ECG, blood pressure | Heart health |
| Skin conductance, potassium, sodium, | Hydration |
| movement | |
| EEG, tremor, eye movement | Brain health |
| Heart rate, movement, GPS, IMU blood | Fitness levels |
| oxygen (SPO2) | |
| Body temperature, cholesterol, blood, | Nutrition quality |
| sugar | |
| Movement, skin conductance, cortisol | Inflammation |
| Audio, blood oxygen | Breathing anomalies |
| Blood alcohol, blood oxygen | Blood health (alcohol, anaemia) |
| Blood Glucose | Specific App 1: Diabetes |
| Temperature, hormones on skin | Specific App 2: Fertility |
| CO2, NO2, CO, air conductivity | Environmental health (air toxins, |
| | humidity) |

These devices can provide very large volumes of specific data—for example, a pedometer device may produce data showing the number of steps taken in a particular period, but does not indicate an angle of ascent of a slope, an altitude that affects air oxygen saturation, and a user's starting, ending and post-exercise resting heart rate. The lack of a cumulative history of multiple factors means that it is difficult to establish a baseline and thus to detect any anomalous readings. It will also not be aware of a user's hydration or blood glucose levels, and so will not have the data to warn the user of possible concerns, such as potential hypo- or hypernatremia or hypo- or hyperglycemia. Thus, while it is possible already to gain information on various biometric measures from the individual devices, the question arises of how to manage and effectively centralize the information from the various biometric sensors to provide an integral view of the health of the user.

The increasing machine learning and inferencing processing capability of smartphones makes these devices a useful tool to accumulate and process data securely to provide real time and continuous monitoring of a user's physical and/or mental (e.g., health) status. Data generated during inferencing operations may, in turn, be used to train or tune a ML model(s) (e.g. neural network(s)) to increase the accuracy of the ML model(s) for subsequent inferencing operations for that user.

Additionally, a phone's own sensors like the camera, microphone and motion sensors can be used as tools to provide data to this process. Implementations of the present technology use smartphones as a hub of a network of health sensors to collect and process securely the information provided. In this way, the hub, responsive to the combined inputs of the devices, is capable of detecting current and future potential health risks and provide a real time physical and/or mental status and health assessment based on machine learning prediction from the accumulated user data. The personal hub, such as a smartphone, may then connect with the user's medical practitioner to share data securely about the preliminary assessment and raise alerts when potential risks are detected. The use of on-device data processing (or secure equivalents thereof) can provide assurances of data privacy and security.

In a further refinement, integrated lab-on-chip technology may be used to process biological samples taken from a user, such as bodily fluids. While biophysical and motion state sensors are available in the market and widely used by consumers, biochemical sampling and analysis devices (which are not so widely available at present), may in future possess significant potential as additional indicators of a user's physical and/or mental status.

In a yet further refinement, geolocation and environmental data from multiple sources, such as GPS satellites and local air quality monitors accessible via, for example, the Internet of Things, may also be used as input to the present technology.

The present technology further provides a possibility of greater accuracy in assessing the health of the user because the varied data from multiple sources can be stored and analysed over time—this enables the production of a comprehensive set of baseline readings for the individual user, which makes the detection of anomalies that may represent risks easier and more accurate. Historic data processing can provide a picture of the user's health in real time, alert the user on immediate and longer-term risks and make recommendations to improve the physical and mental welfare, including nutritional advice.

This “personal general medical practitioner” is not meant to replace a real physician, but can act as complement which is permanently and objectively looking at the user's physical and/or mental status by processing the incoming readings from different sensors in wearable and other devices. The fact that the data processing is performed on the device guarantees its privacy and security as data does not leave the device during processing.

In some implementations, where it is possible either to take the device to a doctor's appointment or to establish secure electronic communications with a medial practitioner, it is also possible to use the present technology as a provider of data to the practitioner. The same technique may further be used to provide data to first responders in emergency situations.

Useful analysis of the plethora of data available from the sensor devices represents a highly complex computational challenge, and the inventors of the present technology have realised that it is desirable to approach this analysis task using artificial intelligence techniques, such as machine learning and inferencing models, possibly implemented in the form of artificial neural networks, to perform these analysis tasks and to provide useful outputs as guidance for a user with respect to maintaining a good physical, mental and health state.

In one concrete example, a wearable sensor may detect a raised body temperature and increased heart rate over a historically determined resting rate, while a pedometer detects a sequence of rapid steps recently taken as in a running action and an epidermal adhesive sensor may detect increased perspiration; at the same time an ingestible device may indicate a low fluid content in the intestine. The analysis based on the ML model may then infer that exercise has led to dehydration, and the technology emits a message suggesting that the user should soon drink a moderate amount of water or to take a rest, as examples of such suggestions.

In particular implementations, ML models may enable improved results in a wide range of tasks, including text, image, video and speech processing, just to provide a couple of example applications. To enable performing such tasks, features of a ML model may be structured and/or configured to form “filters” that may have a measurable/numerical state such as a value of an output signal. Such a filter may comprise nodes and/or edges arranged in “paths” and are to be responsive to sensor observations provided as input signals. In an implementation, a state and/or output signal of such a filter may indicate and/or infer detection of a presence or absence of a feature in an input signal.

In particular implementations, the ML model may comprise one or more neural networks (e.g., nodes, edges, weights, layers of nodes and edges), where intelligent computing devices to perform functions supported by neural networks may comprise a wide variety of stationary and/or mobile devices, such as, for example, smart mobile phones, wearable devices, Internet of things (IoT) devices, personal digital assistants (PDAs), virtual assistants, laptop computers, personal entertainment systems, tablet personal computers (PCs), PCs, just to provide a few examples.

According to an embodiment, a neural network may be structured in layers such that a node in a particular neural network layer may receive output signals from one or more nodes in an upstream layer in the neural network, and provide an output signal to one or more nodes in a downstream layer in the neural network. One specific class of layered neural networks may comprise a convolutional neural network (CNN) or space invariant artificial neural networks (SIANN) that enable deep learning. Such CNNs and/or SIANNs may be based, at least in part, on a shared-weight architecture of a convolution kernels that shift over input features and provide translation equivariant responses. Such CNNs and/or SIANNs may be applied to text, image and/or video recognition, recommender systems, image classification, image segmentation, medical image analysis, natural language processing, brain-computer interfaces, just to provide a few examples.

Another class of layered neural network may comprise a recursive neural network (RNN) that is a class of neural networks in which connections between nodes form a directed cyclic graph along a temporal sequence. Such a temporal sequence may enable modelling of temporal dynamic behavior. In an implementation, an RNN may employ an internal state (e.g., storage (memory)) to process variable length sequences of inputs. This may be applied, for example, to tasks such as unsegmented, connected handwriting recognition or speech recognition, just to provide a few examples. In particular implementations, an RNN may emulate temporal behavior using finite impulse response (FIR) or infinite impulse response (IIR) structures. An RNN may include additional structures to control stored states of such FIR and IIR structures to be aged. Structures to control such stored states may include a network or graph that incorporates time delays and/or has feedback loops, such as in long short-term memory networks (LSTMs) and gated recurrent units.

A neural network (NN) (e.g., CNN, RNN etc.) may have multiple hidden layers in order to model complex, nonlinear relationships between input data and output data, where such neural networks are referred to as deep neural networks (DNN).

A large language model (LLM) which includes a transformer neural network architecture (e.g., decoder-only transformer-based architecture) may be built on one or more DNNs. In implementations of the present technology, such an LLM generates context-aware content in response to the detection of physical and/or mental states by the various biometric sensor devices.

Any suitable storage and processing location may be used to store and process the physical state monitoring data according to the present technology. The storage and processing location may be contained within a single computing-enabled device, such as a smartphone's data processing engine, but the storage and processing location need not be contained wholly within any single device—for example, it may be logically implemented over plural physical locations, such as distributed systems, cloud processing systems, federated data processing systems, and the like.

One or more of the analysis architectures described above may be used to implement the present technology in a computing-capable device such as a personal user device (e.g., a smartphone), although the claims are not limited in this respect.

In an illustrative example, the computing device may include an application that utilizes analysis architecture for processing data (e.g., text data, image data, speech data) generated or received at the computing device. Furthermore, the computing device may utilize plural analysis architectures for analysing two or more different types of data (i.e., multimodal data analysis).

The one or more analysis architecture(s) may, in some examples, be implemented in software and/or data structures, where various nodes, tensors, activation functions, and other elements of processing stages of a neural network may be stored in data structures in storage.

In other examples, the analysis architecture may be implemented in hardware, such as a convolutional neural network structure that is embodied within the transistors, resistors, and other elements of an integrated circuit. In an alternate example, the analysis architecture may be implemented in a combination of hardware and software, such as a neural processing unit (NPU) having software-configurable weights, network size and/or structure, and other such configuration parameters.

The analysis architecture as described herein in particular examples, may be formed in whole or in part by and/or expressed in transistors and/or lower metal interconnects (not shown) in processes (e.g., front end-of-line and/or back-end-of-line processes) such as processes to form complementary metal oxide semiconductor (CMOS) circuitry. The various blocks, neural networks, and other elements disclosed herein may be described using computer aided design tools and expressed (or represented), as data and/or instructions embodied in various computer-readable media, in terms of their behavioral, register transfer, logic component, transistor, layout geometries, and/or other characteristics. Formats of files and other objects in which such circuit expressions may be implemented include, but are not limited to, formats supporting behavioral languages such as C, Verilog, SystemVerilog, and VHDL, formats supporting register level description languages like RTL, and formats supporting geometry description languages such as GDSII, GDSIII, GDSIV, CIF, MEBES and any other suitable formats and languages. Storage media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signalling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.).

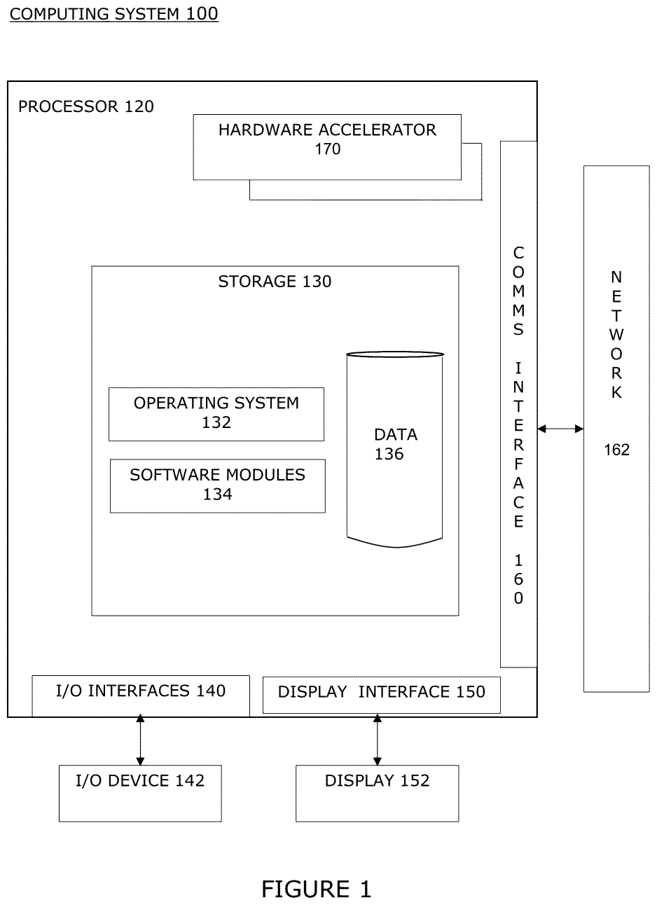

FIG. 1 shows a block diagram of an example computing system 100 which may embody the personal hub technology in accordance with the present techniques. In the present illustrative example, the computing system 100 may comprise a user's personal computing-enabled device (e.g., a smartphone or smart glasses).

Processor 120 may comprise one or more general-purpose or application-specific microprocessors that executes instructions to perform control, computation, input/output, etc. functions for system 100. Processor 120 may include a single integrated circuit, such as a micro-processing device, or multiple integrated circuit devices and/or circuit boards working in cooperation to accomplish the functions of processor 120. Processor 120 may further comprise one or more hardware accelerators (HA) 170 for accelerating processing of specific types of data, such as, for example graphics or audio data.

In an embodiment, processor 120 may be configured to provide the analysis architecture by executing one or more ML models, such as, for example, ANNs, CNNs, RNNs, DNNs, MLPs (Multi-Layer Perceptrons) and LLMs.

Generally, storage (or memory) 130 stores instructions for execution by processor 120 and data. Storage 130 may include a variety of non-transitory computer-readable media that may be accessed by processor 120. In various embodiments, storage 130 may include volatile and non-volatile medium, non-removable medium and/or removable medium. For example, storage 130 may include any combination of random access memory (RAM), dynamic RAM (DRAM), static RAM (SRAM), read only memory (ROM), flash memory, cache memory, and/or any other type of non-transitory computer-readable medium.

Storage 130 may comprise various components for retrieving, presenting, modifying, and storing data. For example, storage 130 may store software modules that provide functionality when executed by processor 120. The software modules include operating system 132 that provides operating system functionality for system 100. Software modules 134 provide various functionality, such as the analysis functionality using one or more neural networks. Data 136 may include data associated with operating system 132, software modules 134, etc.

I/O interfaces 140 are configured to transmit and/or receive data from I/O devices 142. I/O interfaces 140 enable connectivity between processor 120 and I/O devices 142 by encoding data to be sent from processor 120 to I/O devices 142, and decoding data received from I/O devices 142 for processor 120. Generally, data may be sent over wired and/or wireless connections. For example, I/O interfaces 140 may include one or more wired communications interfaces, such as USB, Ethernet, etc., and/or one or more wireless communications interfaces, coupled to one or more antennas, such as Wi-Fi®, Bluetooth®, Zigbee®, Matter, cellular, etc.

Generally, I/O interfaces 140 are coupled to I/O devices 142 using a wired or wireless connection, display interface 150 is coupled to display 152, and communication interface 160 is connected to network 162 using a wired or wireless connection. In many embodiments, certain components of system 100 are implemented as a system-on-chip (SoC) 102; in other embodiments, system 100 may be hosted on a traditional printed circuit board, motherboard, etc.

Generally, I/O devices 142 provide input to processor 120 and/or output from processor 120. As discussed above, I/O devices 142 are operably connected to processor 120 using a wired and/or wireless connection. I/O devices 142 may include a local processor coupled to a communication interface that is configured to communicate with system 100 using the wired and/or wireless connection.

Display interface 150 is configured to transmit image data from system 100 to monitor or display 152.

Communication interface 160 is configured to transmit data to and from network 162 using one or more wired and/or wireless connections. Network 162 may include one or more local area networks, wide area networks, the Internet, etc., which may execute various network protocols, such as, for example, wired and/or wireless Ethernet, Bluetooth®, etc. Network 162 may also include various combinations of wired and/or wireless physical layers, such as, for example, copper wire or coaxial cable networks, fiber optic networks, Bluetooth wireless networks, Wi-Fi® wireless networks, CDMA, FDMA and TDMA cellular wireless networks, etc.

In certain embodiments, the ML model(s) and parameters (e.g. weights) may be stored in non-volatile storage 130 and accessed by software (e.g. applications) executed by the CPU 120 (or one or more HAs 170) via an internal bus (e.g. using direct memory access (DMA)) In other embodiments, the ML model(s) and parameters may be provided from storage 130 for storage in local volatile memory (e.g., local SRAM). In certain embodiments, the ML architecture may be directly implemented in hardware using processing engines, compute engines, matrix multiplier units, MAC arrays, etc.

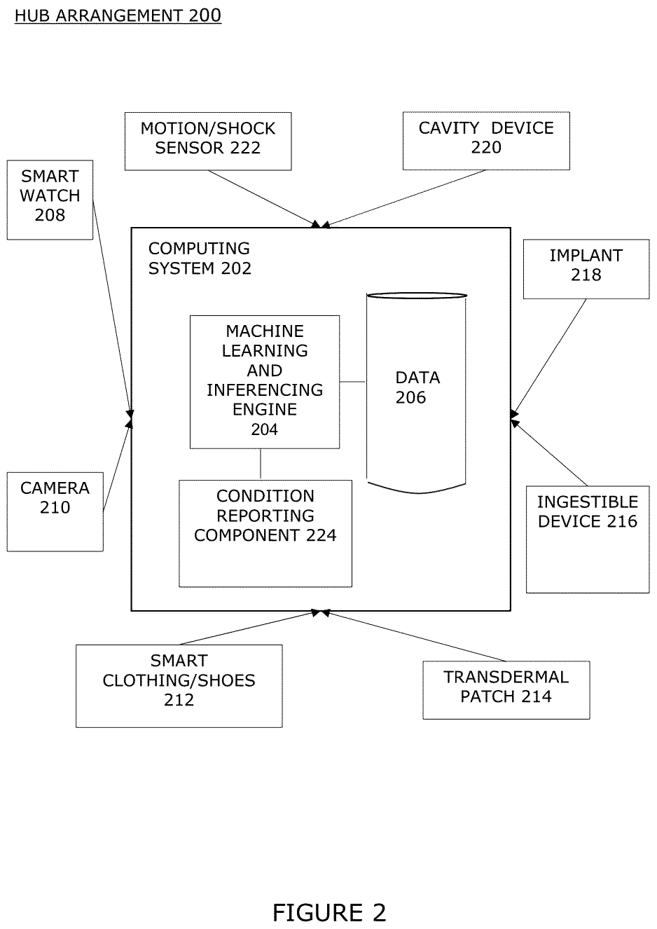

In FIG. 2, there is shown a hub arrangement 200 according to an implementation of the present technology.

Central to the hub arrangement 200 is the computing system 202, which comprises a machine learning and inferencing engine 204 operable to analyse inputs from plural devices, and operable to establish electronic communication with a condition reporting component 224 operable to emit condition information, including risk warnings. The machine learning and inferencing engine 204 is operable to establish electronic communication with data store containing data 206 comprising inputs from plural devices. The computing system 202 is further operable in electronic communication with devices including smart watch 208, camera 210, smart clothing and shoes 212, transdermal patches 214, ingestible devices 216, implants 218, cavity devices 220, motion/shock sensor 222, and the like. Computing system 202 may comprise a secure zone of a processing arrangement. The secure zone may comprise a secure part of a device which has been attested at every stage from boot to provide a trusted region within which security and integrity are guaranteed, or it may comprise a secured and attested portion of a network arrangement, for example a secure realm in a cloud computing environment.

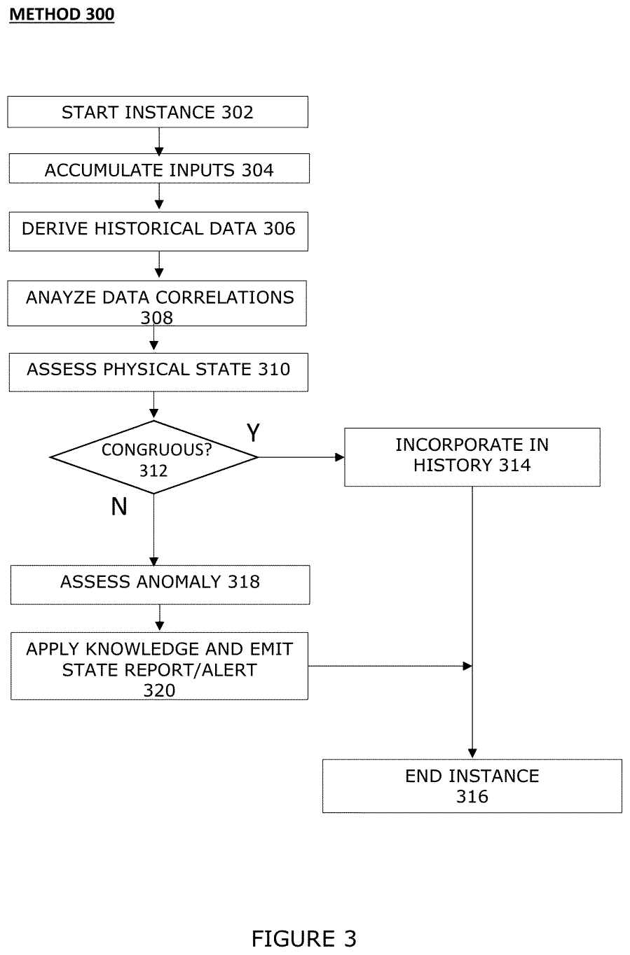

A personal hub according to implementations of the present technology may be operated according to the method 300 as shown in the example FIG. 3. Method 300 begins at START INSTANCE 302. At 304 the technology accumulates inputs from devices that are operable in electronic communication with the personal hub, and at 306 the hub derives historical data from any detected sequences. The hub may establish baseline readings from this historical data by analyzing data correlations at 308 to assess a physical and/or mental state (which may be a baseline state or otherwise) at 310. At 312, the data analysis determines whether a data entity, or a time series of data entities, is congruous with any such baseline.

If it is determined at 312 that the data entity is congruous (that is, it fits into a series or pattern of data entities that may be assumed from the model's knowledge to be normal for that user), the data may be incorporated into the history at 314, and the method 300 completes at END INSTANCE 316. As will be immediately clear to one of ordinary skill in the art, in a real-world implementation, the ending of this instance at 316 may not represent the end of all operations—the method 300 may recommence at an earlier point in the process, including at START INSTANCE 302.

Conversely, if it is determined at 312 that the data entity is not congruous (that is, it does not fit into a series or pattern of data entities that may be assumed from the model's knowledge to be normal for that user), the anomaly is assessed at 318. Typically, the assessment of the anomaly by the ML model takes into account the accumulated knowledge incorporated into the model, as well as the history of the individual user, along with any other relevant factors derived from the user's data entities and time series of data entities to determine whether there is a need to alert the user. Thus at 320, the knowledge embodied in the model is applied to that data and any necessary state report or alert is emitted. The instance then completes at END INSTANCE 316. As will be immediately clear to one of ordinary skill in the art, in a real-world implementation, the ending of this instance at 316 may not represent the end of all operations—the method 300 may recommence at an earlier point in the process, including at START INSTANCE 302.

The examples above describe taking into account the accumulated knowledge incorporated into the model, as well as the history of the individual user, along with any other relevant factors derived from the user's data entities and time series of data entities. The claims are not limited to the data above, and other data about the user may also be used in the analysis. For example, user's age, race, weight, height, body mass index (BMI) gender, location etc. may also be taken into account when determining a condition of a user and, for example, whether there is a need to alert the user.

The ML model is desirably executed in a trusted or protected execution environment (hereafter “trusted execution environment”), where the ML model and associated parameters (e.g., the inputs (e.g., communication data), weights, activation functions, and the outputs) are accessible only to an authorized application or process running on the user device. Such protection may be provided by use of hardware and/or software.

As an illustrative example of a trusted execution environment, communication data may be encrypted, such that only an authorized application or process may decrypt the communication data. As a further example, application data generated by one or more applications running on the system may be stored in storage 130 in a secure manner. For example, such data may be encrypted prior to being stored.

Additionally, or alternatively, in a further illustrative example of a trusted execution environment the computing system 100 may operate with different privilege modes, where each privilege mode can give different rights of access to data. A privilege mode with a higher level of privilege will typically have access to more system resources, where a system resource may be a storage region, peripheral device, more function, data etc.) than are available in a lower privilege mode. Hypervisor code may be provided with a highest privilege mode of operation so as to control access to system resources that are provided to other processes, such as application code, executing on the system, thereby preventing access to system resources (e.g., data) by unauthorized code.

In a further illustrative example of a trusted execution environment, storage access circuitry may be provided to control access to a number of storage regions (e.g., of a memory address space in storage) based on ownership information which defines, for a given storage region, an owner realm specified from among a plurality of realms. Each realm may correspond to at least a portion of at least one software process. The owner realm for a given storage region has the right to exclude other realms from accessing data (e.g., ML models, weights, application data, communication data etc.) stored within the given storage region. Hence, in contrast to a privilege-based model where access permissions may define which processes are allowed to access (read or write) a given storage (memory) region, with the realm-based approach, the owner realm has the ability to control which other realms access its owned storage regions such that different parts of the address space can be allocated different realm owners who have control over access to that part of the address space. Thus, a given realm owner can protect its data from access by other processes including processes operating at the same privilege level or at higher privilege levels.

In some examples, this realm-based approach may be applied in parallel with privilege-based protection model, so that there are multiple overlapping sets of access rights for a given storage region: the privilege-based permissions set by a higher-privilege process, and the access permissions set by the owner realm of the corresponding storage region, where access to data in a particular storage location may be allowed if it satisfies both sets of permissions.

A realm management unit (not shown) may be provided to control operation of a given realm based on security configuration parameters associated with the given realm. For example the security configuration parameters could define information such as a realm type (which may govern what properties the realm has or what operations the realm is able to carry out), a protected address range associated with the given realm (which may mark the bounds of the storage regions which can securely be accessed by the given realm), and other information about whether operation such as debugging or export of data from a storage protector using the storage access circuitry to an external storage outside the bounds of protection by the storage access circuitry would be permitted.

In an implementation of the present technology, a confidential realm may be established such that it can attest to the user or client that the realm environment is secured as expected—that is, that all elements of the realm, such as the bootloader, operating system, and all the processing and data storage resources required are guaranteed to be as claimed and to meet security and integrity requirements. Such a realm is then suitable to be provisioned with the communication data and the model, and the security and integrity of the whole are provided under public key protection via a single secure and attested communications connection.

After the execution of the present technology in a realm as described, any new learning applied to the model may be preserved only by transmitting it to the requesting entity (such as a client or user device) for secure storage at the requesting entity. Any communication and model data, which may potentially expose confidential information, is securely and with a guarantee destroyed either during realm teardown or by any of the known secure explicit deletion mechanisms.

In an illustrative example, the present technology may exploit a trusted execution environment in the form of a private cloud environment, wherein the ML model is operated on a dedicated, isolated processor that is trusted and attested having a single trusted and attested communication channel for communication with the user device.

A suitably trained neural network according to the present technology is thus well suited to the task of efficiently and with resource economy providing secure, intelligent monitoring and assessment of a user's physical and/or mental status and health status.

In a further refinement, there may be arranged a computing environment in which computing devices may communicate suitably protected, anonymised data from a user's device to a federation of devices to contribute to the improvement of the ML models used in the federated devices. The user's data is thus secure, but may, in its anonymised form, help to train models deployed in all the devices in the federation. In the same manner, the anonymised data may assist in epidemiological analysis—for example, if a subset of user devices in the federated arrangement exhibit similar physical and/or mental anomalies with respect to their baselines in close temporal proximity, this may indicate a localised threat to health, such as an outbreak of an infectious or contagious disease, or an environmental contamination event.

A neural network suitable for processing according to the present technology may be trained using supervised training over a data corpus, such as a knowledge base, which training may comprise adapting a generalised neural network or may comprise training a task-specific neural network.

Training a neural network includes optimizing parameters, such as the connection weights between nodes, by minimizing the prediction error of the output data until the neural network achieves a required level of accuracy.

In embodiments, the inputs from plural devices may be assessed such that the data can be prioritised based on the type of the data and the accuracy or trustworthiness of the source of the data. Not all data is equal and some filtering can be useful. In an illustrative example, data from a first source (e.g. a device from a first manufacturer) may have a higher level of trust or determined to be more accurate, and therefore have a higher priority than data from a second source (e.g. a device from a second manufacturer). Furthermore, a device or manufacturer may be blacklisted, and data from a blacklisted device may be discarded. Thus, data can be filtered based on the source of that data.

There are thus provided a computing device and a method of operating a computing device, implementable as a computer program product, where the device may be co-operable with a set of biosensor devices, the method comprising first operating a first transceiver in the computing device to collect a first biosensor device data element into a storage and processing location; second operating the first or a second transceiver to collect a second biosensor device data element into the storage and processing location; applying, in the storage and processing location, machine learning model inferencing over at least the first biosensor device data element and the second biosensor device data element to derive a user condition indication for a biosensor device user; and on detecting, in the user condition indication, a predictive value above a threshold indicating a user condition requiring notification, emitting a message at an output of the computing device, where the user condition requiring notification comprises one or both of a physical and mental condition.

In implementations of the present technology, the storage and processing location for collecting the first biosensor device data element and the second biosensor device data element and applying machine learning model inferencing may be a secure zone of the computing device. In some implementations, the detecting a predictive value above a threshold indicating a physical and/or mental condition requiring notification may comprise identifying in the first biosensor device data element and the second biosensor device data element a combination of values together indicative of the physical and/or mental condition requiring notification. The first biosensor device data element and the second biosensor device data element may be collected from the different biosensor devices or by the same biosensor device in the set of biosensor devices. The first biosensor device data element and the second biosensor device data element may form a time series and the detecting a predictive value above a threshold indicating a physical and/or mental condition requiring notification may be responsive to a change over the time series or to an anomaly with respect to historical data in the time series. Applying machine learning model inferencing over at least the first biosensor device data element and the second biosensor device data element may comprise operating a trained neural network.

The present technology may take the form of a computer program product embodied in a computer readable medium having computer readable program code embodied thereon. The computer readable medium may be a computer readable signal medium or a computer readable storage medium. The computer readable storage medium may be a non-transitory computer readable storage medium encoded with instructions that, when performed by a processing means, cause performance of the method described above. A computer readable medium may be, for example, but is not limited to, an electronic, magnetic, optical, electromagnetic, infrared, or semiconductor system, apparatus, or device, or any suitable combination of the foregoing.

Computer program code for carrying out operations of the present techniques may be written in any combination of one or more programming languages, including object-oriented programming languages and conventional procedural programming languages.

For example, program code for carrying out operations of the present techniques may comprise source, object, or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog™, SystemVerilog, or VHDL (Very high speed integrated circuit Hardware Description Language).

The program code may execute entirely on the user's computer, partly on the user's computer and partly on a remote computer or entirely on the remote computer or server. In the latter scenario, the remote computer may be connected to the user's computer through any type of network. Code components may be embodied as procedures, methods, or the like, and may comprise sub-components which may take the form of instructions or sequences of instructions at any of the levels of abstraction, from the direct machine instructions of a native instruction set to high-level compiled or interpreted language constructs.

It will also be clear to one of skill in the art that all or part of a logical method according to the preferred embodiments of the present techniques may suitably be embodied in a logic apparatus comprising logic elements to perform the steps of the method, and that such logic elements may comprise components such as logic gates in, for example a programmable logic array or application-specific integrated circuit. Such a logic arrangement may further be embodied in enabling elements for temporarily or permanently establishing logic structures in such an array or circuit using, for example, a virtual hardware descriptor language, which may be stored and transmitted using fixed or transmittable carrier media.

In one alternative, an embodiment of the present techniques may be realized in the form of a computer implemented method of deploying a service comprising steps of deploying computer program code operable to, when deployed into a computer infrastructure or network and executed thereon, cause the computer system or network to perform all the steps of the method.

In a further alternative, the preferred embodiment of the present techniques may be realized in the form of a data carrier having functional data thereon, the functional data comprising functional computer data structures to, when loaded into a computer system or network and operated upon thereby, enable the computer system to perform all the steps of the method.

It will be clear to one skilled in the art that many improvements and modifications can be made to the foregoing exemplary embodiments without departing from the scope of the present techniques.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

1. A method of operating a computing device, comprising:
first operating a first transceiver in the computing device to collect a first biosensor device data element into a storage and processing location;
second operating the first or a second transceiver to collect a second biosensor device data element into the storage and processing location;
applying, in the storage and processing location, machine learning model inferencing over at least the first biosensor device data element and the second biosensor device data element to derive a user condition indication for a biosensor device user; and
on detecting, in the user condition indication, a predictive value above a threshold indicating a user condition requiring notification, emitting a message at an output of the computing device, where the user condition requiring notification comprises one or both of a physical and mental condition.

2. The method of claim 1, wherein the storage and processing location for collecting the first biosensor device data element and the second biosensor device data element and applying machine learning model inferencing is a secure zone of the computing device.

3. The method of claim 1, wherein the detecting a predictive value above a threshold indicating a user condition requiring notification comprises identifying in the first biosensor device data element and the second biosensor device data element a combination of values together indicative of the user condition requiring notification.

4. The method of claim 1, wherein the first biosensor device data element and the second biosensor device data element are collected from the same biosensor device.

5. The method of claim 1, wherein the first biosensor device data element and the second biosensor device data element form a time series and the detecting a predictive value above a threshold indicating a user condition requiring notification is responsive to a change over the time series.

6. The method of claim 1, wherein the first biosensor device data element and the second biosensor device data element form a time series and the detecting a predictive value above a threshold indicating a user condition requiring notification is responsive to an anomaly with respect to historical data in the time series.

7. The method of claim 1, wherein applying machine learning model inferencing over at least the first biosensor device data element and the second biosensor device data element comprises operating a trained neural network.

8. The method of claim 1, wherein the collecting of at least one of the first biosensor device data element and the second biosensor device data element comprises collecting data from a wearable device.

9. The method of claim 1, wherein the collecting of at least one of the first data element and the second biosensor device data element comprises collecting data from a sensor integrated into the computing device.

10. The method of claim 9, wherein the collecting data from a sensor integrated into the computing device comprises collecting image data.

11. A computing device comprising:
a transceiver operable to collect a first biosensor device data element into a storage and processing location;
the transceiver or a further transceiver to collect a second biosensor device data element into the storage and processing location;
a machine learning model inferencing engine operable in the storage and processing location to derive a user condition indication for a biosensor device user using at least the first biosensor device data element and the second biosensor device data element; and
an output operable, on detecting in the user condition indication a predictive value above a threshold indicating a user condition requiring notification, to emit a message, where the user condition requiring notification comprises one or both of a physical and mental condition.

12. The computing device of claim 11, wherein the storage and processing location for collecting the first biosensor device data element and the second biosensor device data element and applying machine learning model inferencing is a secure zone of the computing device.

13. The computing device of claim 11, wherein the detecting a predictive value above a threshold indicating a user condition requiring notification comprises identifying in the first biosensor device data element and the second biosensor device data element a combination of values together indicative of the user condition requiring notification.

14. The computing device of claim 11, wherein the first biosensor device data element and the second biosensor device data element are collected from the same biosensor device.

15. The computing device of claim 11, wherein the first biosensor device data element and the second biosensor device data element form a time series and the detecting a predictive value above a threshold indicating a user condition requiring notification is responsive to a change over the time series.

16. The computing device of claim 11, wherein the detecting a predictive value above a threshold indicating a user condition requiring notification is responsive to an anomaly with respect to historical data in the time series.

17. The computing device of claim 11, wherein applying machine learning model inferencing over at least the first biosensor device data element and the second biosensor device data element comprises operating a trained neural network.

18. A computer program product stored on a non-transitory computer readable medium and comprising computer program code to:
first cause a first transceiver to collect a first biosensor device data element into a storage and processing location;
second cause the first or a second transceiver to collect a second biosensor device data element into the storage and processing location;
apply, in the storage and processing location, machine learning model inferencing over at least the first biosensor device data element and the second biosensor device data element to derive a user condition indication for a biosensor device user; and
responsive to detecting, in the user condition indication, a predictive value above a threshold indicating a user condition requiring notification, emitting a message at an output of the computing device, where the user condition requiring notification comprises one or both of a physical and mental condition.

19. The computer program product of claim 18, wherein the collecting the first biosensor device data element and the second biosensor device data element and applying machine learning model inferencing are performed in a storage and processing location in a secure zone of the computing device.

20. A method of operating a computing device, comprising:
determining in an artificial neural network a correlation between at least a first biosensor device data element, a second biosensor device data element, and a physical condition indication for a biosensor device user; and
adjusting the weighting of the artificial neural network according to the correlation to establish a prediction threshold for a physical condition requiring a notification message to be emitted.
